(19) United States
(12) Patent Application Publication
(10) Pub. No.: US 2025/0281093 A1
(43) Pub. Date: Sep. 11, 2025

(54) METHOD FOR DETERMINING THE FUNCTIONAL TOPOGRAPHY OF A PERIPHERAL NERVE

(71) Applicants: SCUOLA SUPERIORE DI STUDI UNIVERSITARI E DI PERFEZIONAMENTO SANT'ANNA, Pisa (IT); ECOLE POLYTECHNIQUE FEDERALE DE LAUSANNE (EPFL), Lausanne (CH)

(72) Inventors: Andrea PITZUS, Quartu Sant'Elena (IT); Simone ROMENI, Ambilly (FR); Fabio VALLONE, Pisa (IT); Silvestro MICERA, Firenze (IT)

(73) Assignees: SCUOLA SUPERIORE DI STUDI UNIVERSITARI E DI PERFEZIONAMENTO SANT'ANNA, Pisa (IT); ECOLE POLYTECHNIQUE FEDERALE DE LAUSANNE (EPFL), Lausanne (CH)

(21) Appl. No.: 18/859,991

(22) PCT Filed: Apr. 26, 2023
(86) PCT No.: PCT/IB2023/054322

§ 371 Date: Oct. 24, 2024

(30) Foreign Application Priority Data
Apr. 27, 2022 (IT) 102022000008381

(51) Int. Cl. A61B 5/294; A61B 5/00; A61B 5/0205; A61B 5/024; A61B 5/318; A61B 5/389

(52) U.S. Cl. CPC A61B 5/294; A61B 5/0205; A61B 5/02416; A61B 5/318; A61B 5/389; A61B 5/6877

(57) ABSTRACT

A method for determining the functional topography of a peripheral nerve (10) of a user comprising the steps of prearranging an electrode (100) comprising a number n of channels cᵢ, with i=1, 2 . . . , n, arranging the electrode (100) in such a way that each channel is in contact with the peripheral nerve (10) at a respective contact point pᵢ, with i=1, 2 . . . , n, generating a model of a cross section S of the peripheral nerve (10) where the area A of the cross section S comprises a number m of areas aⱼ, with j=1, 2, . . . , m, computing a lead field matrix L=[R_{j,i}], wherein R_{j,i} is a value that describes the electrostatic relationship between an area aⱼ and a contact point pᵢ of the cross section S, periodic acquisition, by the electrode (100), of a number n of voltage values Vₖᵢ at instants tₖ, with k=1, 2, . . . , S, obtaining a voltage matrix V=[V_{k,i}], with i=1, 2, . . . , n, where Vₖᵢ is the voltage value determined by the channel cᵢ at the contact point pᵢ at the instant tₖ, periodic acquisition, by at least one medical device, of a number r of values of physiological signals P_{k,h} of the user at instants tₖ, with k=1, 2, . . . , s, obtaining a matrix of the physiological signals P=[P_{k,h}], with h.=1, 2, . . . , r, where Pₖ k is the value of the h-th physiological signal determined at the instant tₖ, computing a discrimination matrix=D=[d_{h,i}], D being function of the matrices V=[V_{k,i}] and P=[P_{k,h}], where d_{h,i} is the discrimination coefficient which represents the correlation between the h-th physiological signal P_{k,h} and the i-th voltage value V_{k,i} referred to a same instant ty computing a spatial filtering matrix Φ^{DBF}=[φ_{h,j}], φ_{k,j} being the localization index which represents the correlation between the h-th physiological signal and the area aⱼ of said cross section S, generating a functional topography of said peripheral nerve (10), for each h-th physiological signal, wherein each area aⱼ, is graphically identified as a function of the corresponding value φ_{h,j} associated with it by the spatial filtering matrix Φ^{DBF}.

### FIELD OF THE INVENTION

The present invention relates to the field of neural activity mapping.

In particular, the present invention relates to the determination of the functional topography of a peripheral nerve for the automatic optimization of spatially selective stimulation protocols with respect to multiple functions.

### DESCRIPTION OF THE PRIOR ART

The autonomic nervous system (ANS) is the part of the peripheral nervous system that interacts with the visceral organs to guarantee the chemical-physical balance (homeostasis) of the organism. The ANS communicates with vital organs including the heart, lungs and digestive tract via nerves, made up of bundles of nerve fibers called fascicles dispersed in a matrix of connective tissue. The “topography” of a nerve is defined as the spatial organization of the fascicles in its cross section.

The electrical stimulation of a nerve makes it possible to modulate the activity of the organs or to transmit sensory stimuli to the brain, which interprets them as coming from the target organs of the innervation. Electrical stimulation is performed using electrodes that are surgically applied to the nerve and placed in contact with the outer surface of the nerve (extra-neural electrodes) or inserted into the section of the nerve (intra-neural electrodes). Stimulation is delivered by activating the different conductive contacts of the electrode over time, which cause some electrical activity in the nerve. This variation over time of the currents injected or absorbed by the electrode contacts is called the stimulation protocol. An electrical stimulation protocol acts selectively on a function of the organism if it is capable of significantly modifying this function without altering the other functions controlled by the nerve under stimulation. Since the autonomic nervous system interacts with a large number of vital organs that perform completely heterogeneous functions, non-selective stimulation can produce even serious unwanted effects.

The scientific community has shown in the past that nerve fibers that interact with a given function often exhibit a certain level of spatial segregation compared to fibers that interact with different functions. The spatial organization of fibers related to different body functions is called functional topography, and its determination in the least invasive way possible allows the development of spatially selective stimulation protocols.

By its constitution, the flow of nervous activity that crosses the autonomic system is generated by the simultaneous activity of multiple sources of information. The overall nervous activity crossing a nerve can be recorded using the same electrodes that can be used for stimulation, and therefore without the need to implant additional devices. Nervous activity originating or destined for vital organs such as the heart or lungs can then be related to physiological signals normally recorded in a non-invasive way, such as blood pressure.

The existing methods of reconstruction of the functional topography of a nerve, based on the use of the electroneurographic signal (ENG), are based on the concepts of lead field matrix and of discriminability index.

The process of recording an ENG signal, i.e. the electrical activity produced in the cross section of a nerve, can be simulated by calculating a value of the current injected by each segment of each fiber present in the nerve and a constant which relates this segment with the registration contact of the electrode. The constant that establishes the contribution of each fiber in the overall recording can be calculated by finite element analysis (FEA or FEM), so that membrane currents of fibers far from the recording contact have a smaller contribution within the recorded signal. These constants are characteristic of a given implant (geometry of the implanted nerve and electrode) and must be calculated only once for each application. The constants relating to each recording contact in the implanted electrode (which can be thought of as the “fields of vision” of each contact) are usually collected in several rows of a matrix, called the lead field matrix. Each column of the lead field matrix refers to a single fiber and contains the contribution of this fiber to the signals recorded by each contact of the electrode used. The pseudo-inverse of the lead field matrix has as many columns as there are contacts of the electrode used, and each of these columns represents a spatial filter that converts the signal recorded by a specific contact into a distribution of electrical power in each point of the nerve.

Document US20110046506A1 describes a triangulation method by spatial filtering (“beamforming”, BF), which uses these fields of vision by weighing them by the power of the signal recorded by each contact. Thus, the localization of nerve transmitted power is a weighted sum of the spatial filters for each contact, where contacts registering higher power signals have a higher contribution, since they are likely closer to the source of the power.

However, in this document the localization procedure does not allow the components relating to a physiological function to be selected and filtered. This can result in off-target activation by electrical stimulation, producing adverse effects that can cause severe discomfort to the patient.

In the document F. Vallone et al., “Simultaneous decoding of cardiovascular and respiratory functional changes from pig intraneural vagus nerve signals” J. Neural Eng., vol. 18, no. 4, p. 0460a2, July 2021, doi: 10.1088/1741-2552/ac0d42 a method is described for discrimination through potential fields (“discriminative field potential”, DFP) which directly uses the lead field matrix. The lines corresponding to each site are weighted with respect to a discriminability coefficient and added together to produce a spatial distribution with higher values in the areas where it is more probable that a given information source is present.

However, since in this document the inversion of the lead field matrix is not carried out, there is no triangulation: the information coming from the individual contacts is used without eliminating the redundancy due to the fact that the contacts register the same signal from certain locations in the section of the nerve. The localization is therefore limited to the identification of the zones of influence of the most informative contacts, and there is no construction of a real functional topography of the nerve.

In the document F. Vallone et al., the discriminability coefficient is defined as a number that is assigned to each contact of the electrode and quantifies the information related to a certain physiological state induced in a subject represented in the ENG signal.

In the context of such invention, more specifically, the discriminability coefficient is defined as a number that is assigned to each contact of the electrode and quantifies the information related to a certain physiological function present in the ENG signal.

### SUMMARY OF THE INVENTION

It is therefore a feature of the present invention to provide a method for determining the functional topography of a peripheral nerve of a user which allows to avoid the stimulation of unwanted components and therefore the production of severe adverse effects for the patient.

It is also a feature of the present invention to provide such a method which allows to accurately determine the spatial/topographical organization in the nerve section of the nerve fibers communicating with different organs which mediate for different functions of the organism.

It is still a feature of the present invention to provide such a method which allows the relationship between nerve signals and physiological measurements to be used to triangulate the position in the section of the nerve of the fibers which transmit the information relating to each information source.

These and other objects are achieved by a method for determining the functional topography of a peripheral nerve of a user, said method requiring an electrode (100) comprising a number n of channels cᵢ, with i=1, 2, . . . , n, wherein each channel cᵢ is in contact with said peripheral nerve at a respective contact point pᵢ, with i=1, 2, . . . , n, said method comprising the steps of:
generating a model of a cross section S of said peripheral nerve where the area A of said cross section S comprises a number m of areas aⱼ, with j=1, 2, . . . , m;
computing a lead field matrix L=[R_{j,i}], wherein R_{j,i} is a value that describes the electrostatic relationship between an area aⱼ and a contact point pᵢ of said cross section S;
periodic acquisition, by said electrode (100), of a number n of voltage values V_{k,i} at instants tₖ, with k=1, 2, . . . , s, obtaining a voltage matrix V=[V_{k,i}], with i=1, 2, . . . , n, where V_{k,i} is the voltage value determined by the channel cᵢ at the contact point pᵢ at the instant tₖ;
periodic acquisition, by at least one medical device, of a number r of values of physiological signals P_{k,h} of said user at instants tₖ, with k=1, 2, . . . , s, obtaining a matrix of the physiological signals P=[P_{k,h}], with h=1, 2, . . . , r, where P_{k,h} is value of the h-th physiological signal determined at the instant tₖ;
computing a discrimination matrix D=[d_{h,i}], D being function of said matrix V=[V_{k,i}] and P=[P_{k,h}], where d_{h,i} is the discrimination coefficient which represents the correlation between the h-th physiological signal P_{k,h} and the i-th voltage value V_{k,i} referred to a same instant tₖ;
computing a spatial filtering matrix ϕ^{DBF}=[φ_{h,j}], φ_{h,j} being the localization index which represents the correlation between the h-th physiological signal and the area aⱼ of said cross section S;
for each h-th physiological signal, generating a functional topography of said peripheral nerve wherein each area aⱼ is graphically identified as a function of the corresponding value φ_{h,j} associated with it by said spatial filtering matrix ϕ^{DBF}.

The present invention therefore provides a method of discriminative triangulation by spatial filtering (“discriminative beamforming”, DBF) which uses the discriminability coefficient to weight the spatial filters used in the triangulation and thus take into account the relative positions of the contacts in the recording electrode. This method makes it possible to obtain a localization measure in the proper sense and to select and filter the components relating to a physiological function by means of the discrimination coefficients.

In particular, by means of the electrode it is possible to produce currents in the channels cᵢ such that in correspondence with the desired areas aⱼ these currents produce a significant effect on the physiological parameters correlated to them.

Advantageously, a step is also provided of filtering said voltage matrix V=[V_{k,i}] obtaining a filtered voltage matrix V̌=[V̌_{k,i}]=filt (V).

Advantageously, a step is also provided of extracting features from said filtered voltage matrix V̌=[V̌_{k,i}] obtaining a neural data matrix X_{ENG}.

In particular, said discrimination matrix D=[d_{h,i}] is function of said neural data matrix X_{ENG}.

In particular, the step of filtering said voltage matrix V=[V_{k,i}] is obtained by the equation:

$${\check{V}}_{k , i} = filt ( {V}_{k , i} ) = {V}_{k , i} * {H}_{k} = {\sum }_{τ = 0}^{τ = L} {V}_{τ , i} \cdot {H}_{k - τ}$$

where Hₖ is value of the k-th coefficient, with k=1, 2, . . . , L, of the (impulse) response of the filter.

Alternatively, the step of filtering said voltage matrix V=[V_{k,i}] is obtained by the equation:

$${\check{V}}_{k , i} = zscore ( {V}_{k , i} ) = \frac{{V}_{k , i} - {η}_{{V}_{k , i}}}{{σ}_{{V}_{k , i}}}$$

where η_{V}_{ₖ}_{,i}e φV_{k,i} represent respectively the mean and the standard deviation of the i-th neural datum V_{k,i}.

Alternatively, the step of filtering said voltage matrix V=[V_{k,i}] is obtained by the equation:

$${\check{V}}_{k , i} = abs ( {V}_{k , i} ) = \vert {V}_{k , i} \vert$$

Alternatively, the step of filtering said voltage matrix V=[V_{k,i}] is obtained by the equation:

$${\check{V}}_{k , i} = RMS ( {V}_{k , i} ) = \frac{1}{K} {\sum }_{τ = k - dk}^{τ = k + dk} {V}_{τ , i}^{2}$$

where K represents the number of values taken into consideration.

In particular, said step of filtering said voltage matrix V=[V_{k,i}] comprises the steps of:
for each channel cᵢ, defining a set Gᵢ comprising all the voltage values V_{k,i} taken at said channel cᵢ;
applying a filter on said set Gᵢ, obtaining a filtered set Ǧᵢ comprising filtered voltage values V̌_{k,i};
obtaining a filtered voltage matrix V̌=[V̌_{k,i}]=filt (V).

In particular, said step of extracting features from said filtered voltage matrix V̌=[V̌_{k,i}] comprises the steps of:
defining a time window Δt_{\tilde{k}}=b*Δtₖ, with Δt_{\tilde{k}}=(t_{\tilde{k}+1}−t_{\tilde{k}}) and Δtₖ=(tₖ₊₁−tₖ), where b≥1 is a predetermined coefficient;
for each filtered set Ǧᵢ, selection of filtered voltage values V̌_{k,i} acquired in said time window Δt_{\tilde{k}}, obtaining a number s/b of subsets \tilde{G}_{k,i}, with K=1, 2, . . . , s/b, each subset \tilde{G}_{k,i} comprising a number b of filtered voltage values V̌_{k,i};
for each subset \tilde{G}_{k,i}, extraction of a number f of neural data arranged to define mathematical features of said subset \tilde{G}_{k,i}, obtaining a number n*f of neural data for each filtered set Ǧᵢ;
obtaining a neural data matrix X_{ENG}=[\tilde{V}_{k,i}], where \tilde{V}_{k,i} is the ĩ-th neural datum extracted in the window Δt_{\tilde{k}}, ĩ=1, 2, . . . , n*f.

In particular, a mathematical feature extracted from said subset \tilde{G}_{k,i} is the local maximum defined by the equation:

$${\tilde{V}}_{\tilde{k} , \tilde{ι}} = {local max}_{{t}_{\tilde{k}} < τ < {t}_{\tilde{k} + 1}} {\check{V}}_{τ , i} = local max {\tilde{G}}_{\tilde{k} , i}$$

In particular, a mathematical feature extracted from said subset \tilde{G}_{k,i} is the local minimum defined by the

$${\tilde{V}}_{\tilde{k} , \tilde{ι}} = {local min}_{{t}_{\tilde{k}} < τ < {t}_{\tilde{k} + 1}} {\check{V}}_{τ , i} = local min {\tilde{G}}_{\tilde{k} , i}$$

In particular, a mathematical feature extracted from said subset \tilde{G}_{k,i} is the number of values above the threshold defined by the relation:

$${\tilde{V}}_{\tilde{k} , \tilde{ι}} = {\#}_{{t}_{\tilde{k}} < τ < {t}_{\tilde{k} + 1}} {\check{V}}_{τ , i} \geq {thr}_{i} = \# {\tilde{G}}_{\tilde{k} , i} \geq {thr}_{i}$$

where thrᵢ is the threshold value of the i-th filtered set Ǧᵢ.

Advantageously, a step is also provided of filtering said matrix of the physiological signals P=[P_{k,h}] obtaining a filtered matrix of the physiological signals P̌=[P̌_{k,h}]=filt(P).

Advantageously, a step is also provided of extracting features from said filtered matrix of the physiological signals P̌=[P̌_{k,h}] obtaining a functional data matrix X_{PHYSIO}.

In particular, said discrimination matrix D=[d_{h,i}] is function of said neural data matrix X_{PHYSIO}.

In particular, the step of filtering said matrix of the physiological signals P=[P_{k,h}] is obtained by the equation:

$${\check{P}}_{k , h} = filt ( {P}_{k , h} ) = {P}_{k , h} * {H}_{k} = {\sum }_{τ = 0}^{τ = L} {P}_{τ , h} \cdot {H}_{k - τ}$$

where Hₖ is value of the k-th coefficient, with k=1, 2, . . . , L, of the (impulse) response of the filter.

Alternatively, the step of filtering said matrix of the physiological signals P=[P_{k,h}] is obtained by the equation:

$${\check{P}}_{k , h} = zscore ( {P}_{k , h} ) = \frac{{P}_{k , h} - {η}_{{P}_{k , h}}}{{σ}_{{P}_{k , h}}}$$

where η_{P}_{_{k,h}}e σP_{k,h} represent respectively the mean and the standard deviation of the h-th functional datum P_{k,h}.

Alternatively, the step of filtering said matrix of the physiological signals P=[P_{k,h}] is obtained by the equation:

$${\tilde{P}}_{k , h} = abs ( {P}_{k , h} ) = \vert {P}_{k , h} \vert$$

Alternatively, the step of filtering said matrix of the physiological signals P=[P_{k,h}] is obtained by the equation:

$${\check{P}}_{k , h} = RMS ( {P}_{k , h} ) = \sqrt{\frac{1}{K} {\sum }_{τ = k - d k}^{τ = k + d k} {P}_{τ , h}^{2}}$$

where K represents the number of values taken into consideration.

In particular, said step of filtering said matrix of the physiological signals P=[P_{k,h}] comprises the steps of:
for each h-th physiological signal, defining a set Gₕ comprising all the values of said h-th physiological signal P_{k,h} acquired;
applying a filter on said set Gₕ, obtaining a filtered set Ǧₕ comprising values of the filtered physiological signals P̌_{k,h};
obtaining a filtered matrix of the physiological signals P̌=[P̌_{k,h}]=filt(P).

In particular, said step of extracting features from said filtered matrix of the physiological signals P̌=[P̌_{k,h}] comprises the steps of:
defining a time window Δt_{\tilde{k}}=b*Δtₖ, with Δt_{\tilde{k}}=(t_{\tilde{k}+1}−t_{\tilde{k}}) and Δtₖ=(tₖ₊₁−tₖ), where b≥1 is a predetermined coefficient;
for each filtered set Ǧₕ, selection of values of filtered physiological signals P̌_{k,h} acquired in said time window Δtₖ, obtaining a number s/b of subsets \tilde{G}_{k,h}, with \tilde{k}=1, 2, . . . , s/b, each subset \tilde{G}_{k,h} comprising a number b of filtered physiological signals P̌_{k,h};
for each subset \tilde{G}_{k,h}, extraction of a number w of functional data arranged to define mathematical features of said subset \tilde{G}_{k,h}, obtaining a number n*w of functional data for each filtered set Ǧₕ;
obtaining a functional data matrix X_{PHYSIO}=[\tilde{P}_{k,h}], where \tilde{P}_{k,h} is the \tilde{h}-th functional datum extracted in the window Δt_{\tilde{k}}, \tilde{h}=1, 2, . . . , n*w.

In particular, a mathematical feature extracted from said subset \tilde{G}_{k,h} is the local maximum defined by the equation:

$${\tilde{P}}_{\tilde{k} , \tilde{h}} = {local max}_{{t}_{\tilde{k}} < τ < {t}_{\tilde{k} + 1}} {\check{P}}_{τ , h} = local max {\tilde{G}}_{\tilde{k} , h}$$

In particular, a mathematical feature extracted from said subset \tilde{G}_{k,h} is the local minimum defined by the

$${\tilde{P}}_{\tilde{k} , \tilde{h}} = {local min}_{{t}_{\tilde{k}} < τ < {t}_{\tilde{k} + 1}} {\check{P}}_{τ , h} = local min {\tilde{G}}_{\tilde{k} , h}$$

In particular, a mathematical feature extracted from said subset \tilde{G}_{k,h} is the number of values above the threshold defined by the relation:

$${\tilde{P}}_{\tilde{k} , \tilde{h}} = {\#}_{{t}_{\tilde{k}} < τ < {t}_{\tilde{k} + 1}} {\check{P}}_{τ , h} \geq {thr}_{h} = \# {\tilde{G}}_{\tilde{k} , h} \geq {thr}_{h}$$

where thrₕ is the threshold value of the i-th filtered set Ǧₕ.

In particular, said step of computing said discrimination matrix D is obtained solving the system:

$$\{ \begin{matrix}{X}_{ENG} = {X}_{PHYSIO} D + ε \\ D = {( {X}_{PHYSIO}^{T} {C}_{ε}^{- 1} {X}_{PHYSIO} )}^{- 1} {X}_{PHYSIO}^{T} {C}_{ε}^{- 1} {X}_{ENG} \\ {C}_{ε} = E \{ ( ε - {η}_{ε} ) {( ε - {η}_{ε} )}^{T} \}\end{matrix}$$

where C_{ε} is the error covariance matrix,
E is the expected value operator,
ε is the error matrix in which the residuals of the predictive model are present.

In particular, ε=[ε_{\tilde{k},ĩ}] is the matrix error wherein ε_{\tilde{k},ĩ} is value of the error in the prediction of the i-th neural datum of X_{ENG} at the instant t_{\tilde{k}}.

The above-described equation is valid if there is linear correlation between each value P_{k,h} and a corresponding value V_{k,i} referred to a same instant tₖ.

Alternatively, said step of computing said discrimination matrix D is obtained by the equation:

$${d}_{h , i} = corr ( {X}_{{PHYSIO}_{k , h}} , {X}_{{ENG}_{k , i}} ) = \frac{{σ}_{{X}_{{PHYSIO}_{k , h}} , {X}_{{ENG}_{k , i}}}}{{σ}_{{X}_{{PHYSIO}_{k , h}}} {σ}_{{X}_{{ENG}_{k , i}}}}$$

where σ_{X}_{_{PHYSIOk,h}}_{X}_{_{ENGk,i}} is the covariance of the variables X_{PHYSIO}_{_{k,h}} and X_{ENG}_{_{k,i}},
σ_{X}_{_{PHYSIOk,h}}/σ_{X}_{_{ENGk,i}} is the standard deviation of X_{PHYSIO}_{_{k,h}}/X_{ENG}_{_{k,i}}.

The above-described equation is valid if there is linear correlation between each value P_{k,h} and a corresponding value V_{k,i} referred to a same instant tₖ.

In particular, said step of computing said spatial filtering matrix ϕ^{DBF} is obtained according to the equation:

ϕ^{DBF}=DL⁺
with L⁺=(L^{T}L)⁻¹L^{T}.

Alternatively, said step of computing said spatial filtering matrix ϕ^{DBF} is obtained according to the equation:

ϕ^{DBF}=DL_{Λ}⁺
with L_{Λ}⁺=(L^{T}ΛL)⁻¹L^{T}Λ,
where Λ=[Λ_{j,j}] is the spatial information matrix, being Λ_{j,j}=1 when it is known that the area aⱼ corresponds to a nonzero value of φ_{h,j}.

Alternatively, said step of computing said spatial filtering matrix ϕ^{DBF} is obtained according to the equation:

$${ϕ}^{DBF} = D {\hat{L}}_{Λ}^{+}$$

$$with {L}_{Λ}^{+} = {( {L}^{T} Λ L )}^{- 1} {L}^{T} Λ and {\hat{L}}_{Λ}^{+} [ : , j ] ← \frac{{\hat{L}}_{Λ}^{+} [ : , j ]}{{‖ Λ {LL}_{Λ}^{+} [ : , j ] ‖}_{2}} ,$$

where Λ=[Λ_{j,j}] is the spatial information matrix, being Λ_{j,j}=1 when it is known that the area aⱼ corresponds to a nonzero value of φ_{h,j}.

Advantageously, if there is no linear correlation between each value P_{k,h} and a corresponding value V_{k,i} referred to the same instant tₖ, said step of computing said discrimination matrix D is obtained by at least one of the following techniques:
calculation of advanced similarity metrics;
indices relating to information theory (e.g. mutual information) or to complexity analysis;
fuzzy logic techniques;
artificial intelligence techniques (e.g. neural networks).

In particular, the step of computing the discrimination matrix D is obtained by the technique of mutual information by the equation:

$${d}_{h , i} = {\sum }_{{X}_{{PHYSIO}_{\tilde{k} , \tilde{h}}}} {\sum }_{{X}_{{ENG}_{\tilde{k} , \tilde{ι}}}} {p}_{{X}_{{PHYSIO}_{\tilde{k} , \tilde{h}} , {X}_{{ENG}_{\tilde{k} , \tilde{ι}}}}} log ( \frac{{p}_{{X}_{{PHYSIO}_{\tilde{k} , \tilde{h}} , {X}_{{ENG}_{\tilde{k} , \tilde{ι}}}}}}{{p}_{{X}_{{PHYSIO}_{\tilde{k} , \tilde{h}}}} {p}_{{X}_{{ENG}_{\tilde{k} , \tilde{ι}}}}} )$$

where \tilde{h}∈h, ĩ∈i, p_{X}_{_{PHYSIO\tilde{k},\tilde{h}}}_{X}_{_{ENG\tilde{k},ĩ}} is the joint probability distribution function of X_{PHYSIO}_{_{\tilde{k},\tilde{h}}} and X_{ENG}_{_{\tilde{k},ĩ}},
p_{X}_{_{PHYSIO\tilde{k},\tilde{h}}} and p_{X}_{_{ENG\tilde{k},ĩ}} are the marginal probability distribution functions, respectively, of X_{PHYSIO\tilde{k},\tilde{h}} and X_{ENG\tilde{k},ĩ}.

In particular, the step of computing the discrimination matrix D is obtained by the neural networks by the relations:

$${X}_{PHYSIO} \approx Ψ ( {X}_{ENG} W - b ) + ε$$

$$D ∝ W , b , {ε}^{- 1}$$

where W=[w_{i,u}] is the weight matrix,
ε is the error matrix in which the residuals of the predictive model are present,
b=[b_{i,u}] is the matrix of bias coefficients, with u=1, 2, . . . , q, where w_{i,u} and b_{i,u} are the values of the i-th neural datum and of the n-th artificial neuron,
Ψ is the activation function and acts as an approximator of functions, taking the form of a universal function (e.g. tanh, radial basis, etc.).

Advantageously, said step of generating a functional topography of said peripheral nerve is obtained by associating a plurality of numerical ranges of said values φ_{h,j} to respective colours or colour shades. Alternatively, said step of generating a functional

topography of said peripheral nerve is obtained adopting the technique of field lines (or isolines) to said spatial filtering matrix ϕ^{DBF} in order to delineate in said cross section S of the peripheral nerve a perimeter Π containing the areas aⱼ correlated to a given physiological parameter.

Alternatively, said step of generating a functional topography of said peripheral nerve is obtained by assigning to each area aⱼ a value of statistical significance, obtained by carrying out a statistical test (e.g. t-test), with respect to the activation of this area in relation to the activation of a given physiological parameter.

In particular, the acquired physiological signals comprise, alternatively or in combination:
electrophysiological signals that measure the response of the autonomic nervous system: electrocardiogram (ECG), electromyogram (EMG), galvanic response of the skin;
vital signs of the autonomic nervous system associated with the cardiovascular and respiratory systems: blood pressure, lung volume or other respiratory signals, oxygen saturation;
other signals capable of measuring the response of the autonomic nervous system: blood glucose level, body temperature.

In particular, the medical device is selected from the group consisting of:
wearable or non-wearable electromedical systems for the acquisition of bio-potentials and/or bio-impedances;
wearable or non-wearable devices for detecting pressure and blood values: photo-plethysmograph, pulse oximeter, electronic meter based on the oscillometric method;
wearable and non-wearable devices for breath detection: spirometer and devices for mechanical ventilation, impedance or inductance plethysmograph, piezo-resistive or piezo-electric pneumograph, thermo-couple;
manual or automatic wearable or non-wearable (needle) blood glucose monitoring systems;
wearable or non-wearable body temperature monitoring systems: thermo-couple thermometer, resistive sensor thermometer, infrared thermometer.

### BRIEF DESCRIPTION OF THE DRAWINGS

The invention will be now shown with the following description of its embodiments, exemplifying but not limitative, with reference to the attached drawings in which:

FIG. 1 shows a flow diagram of the successive steps of the method according to the present invention;

FIG. 2 schematically shows an electrode applied to a peripheral nerve;

FIG. 2A schematically shows a section of the peripheral nerve to which a first embodiment of the electrode is applied;

FIG. 2B schematically shows a section of the peripheral nerve to which a second embodiment of the electrode is applied;

FIG. 3 shows a possible graphical visualization of the functional topography of the peripheral nerve obtained by the method according to the present invention;

FIG. 4A schematically shows the process of stimulation, by means of the electrode, of an area of the peripheral nerve assigned to the desired target function;

FIG. 4B schematically shows the connection between the vagus nerve and some organs responsible for physiological functions which can be stimulated electrically thanks to the functional topography of the nerve obtained by means of the method of the present invention.

### DESCRIPTION OF SOME PREFERRED EMBODIMENTS

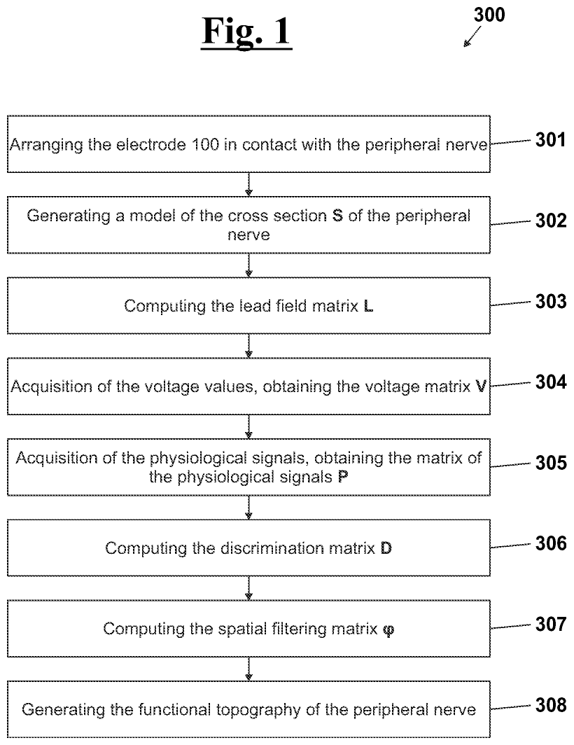

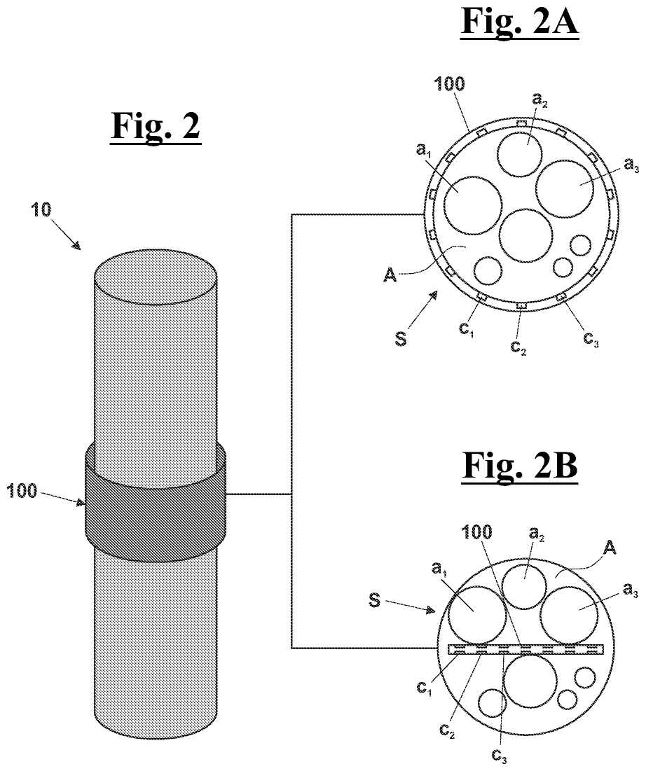

With reference to FIGS. 1, 2, 2A and 2B, the method for determining the functional topography of a peripheral nerve 10 of a user, according to the present invention, provides a first step of arranging an electrode 100 comprising a number n of channels cᵢ, with i=1, 2, . . . , n, in contact with the peripheral nerve 10. In particular, the electrode 100 is arranged in such a way that each channel cᵢ is in contact with the peripheral nerve 10 at a respective contact point pᵢ, with i=1, 2, . . . , n.

In particular, in FIG. 2A a first embodiment is shown of the electrode 100 where the channels cᵢ are arranged in contact with the outer surface of the peripheral nerve 10, whereas in FIG. 2B an alternative embodiment is shown where the channels cᵢ are arranged internally to the section of the peripheral nerve 10.

The method then provides a step of generating a model of a cross section S of the peripheral nerve 10 where the area A of the cross section S comprises a number m of areas aⱼ, with j=1, 2, . . . , m. This model, which also includes the electrode 100, allows the subsequent calculation steps of the method.

The method then provides a step of computing a lead field matrix L=[R_{j,i}], wherein R_{j,i} is a value that describes the electrostatic relationship between an area aⱼ and a contact point pᵢ of said cross section S [303]. Such values R_{j,i} depend on the relative spatial arrangement between the peripheral nerve 10 and the electrode 100 and therefore depend on the specific geometry of the electrode 100.

The method then provides a step of periodic acquisition, by the electrode 100, of a number n of voltage values V_{k,i} at instants tₖ, with k=1, 2, . . . , s, obtaining a voltage matrix V=[V_{k,i}], with i=1, 2, . . . , n, where V_{k,i} is the voltage value determined by the channel cᵢ at the contact point pᵢ at the instant tₖ.

The method also comprises a step of periodic acquisition, by at least one medical device, of a number r of values of physiological signals P_{k,h} of the user at instants tₖ, with k=1, 2, . . . , s, obtaining a matrix of the physiological signals P=[P_{k,h}], with h=1, 2, . . . , r, where P_{k,h} is value of the h-th physiological signal determined at the instant tₖ.

In particular, the medical device, not shown in the figures for simplicity's sake, can be for example a device for acquiring electrophysiological signals that measure the response of the autonomic nervous system or vital signs of the autonomic nervous system associated with the cardiovascular and respiratory systems.

Once the matrices V=[V_{k,i}] and P=[P_{k,h}] have been obtained, the method provides a step of computing a discrimination matrix where D=[d_{h,i}], d_{h,i} is the discrimination coefficient which represents the correlation between the h-th physiological signal P_{k,h} and the i-th voltage value V_{k,i} referred to the same instant tₖ. The discrimination matrix D thus allows to evaluate the degree of correlation between each channel cᵢ of the electrode 100 and each h-th physiological signal.

In particular, if there is a linear correlation between each value P_{k,h} and a corresponding value V_{k,i} referred to the same instant tₖ, the discrimination matrix D is obtained solving the system:

$$\{ \begin{matrix}{X}_{ENG} = {X}_{PHYSIO} D + ε \\ D = {( {X}_{PHYSIO}^{T} {C}_{ε}^{- 1} {X}_{PHYSIO} )}^{- 1} {X}_{PHYSIO}^{T} {C}_{ε}^{- 1} {X}_{ENG} \\ {C}_{ε} = E \{ ( ε - {η}_{ε} ) {( ε - {η}_{ε} )}^{T} \}\end{matrix}$$

where C_{ε} is the error covariance matrix,
E is the expected value operator,
ε is the error matrix in which the residuals of the predictive model are present.

The method then provides a step of computing a spatial filtering matrix ϕ^{DBF}=[φ_{h,j}], φ_{h,j} being the localization index which represents the correlation between the h-th physiological signal and the area aⱼ of the cross section S. The spatial filtering matrix ϕ^{DBF} therefore makes it possible to evaluate which area aⱼ of the cross section S assigned to the modulation of the h-th parameter physiological.

The method then provides, for each h-th physiological signal, a step of generating a functional topography of the peripheral nerve 10 wherein each area aⱼ is graphically identified as a function of the corresponding value φ_{h,j} associated with it by the spatial filtering matrix ϕ^{DBF} [308].

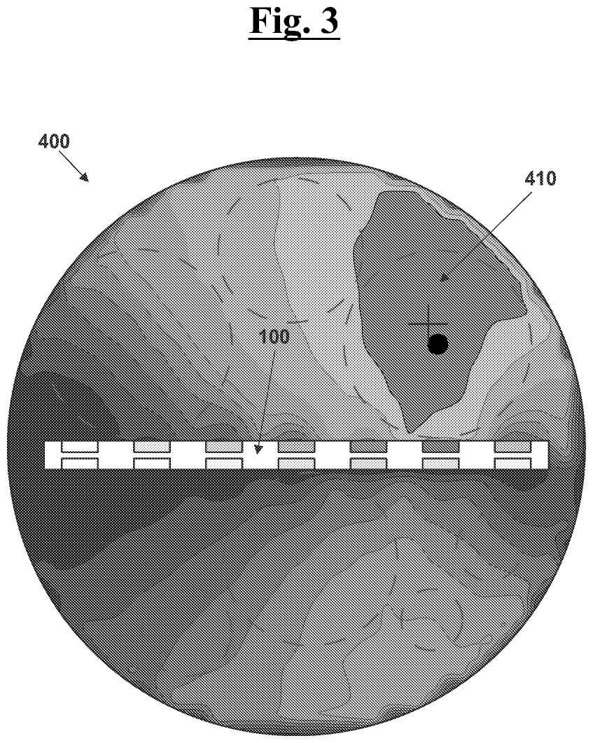

With reference even at FIG. 3, the graphic visualization 400 of the functional topography can be obtained by associating a plurality of numerical ranges of the values φ_{h,j} to respective colours or colour shades. Furthermore, the graphic visualization 400 can be made more intuitive by adopting the technique of field lines (or isolines) in order to outline in the cross section S of the peripheral nerve 10 a perimeter Π containing the areas aⱼ correlated to a given physiological parameter.

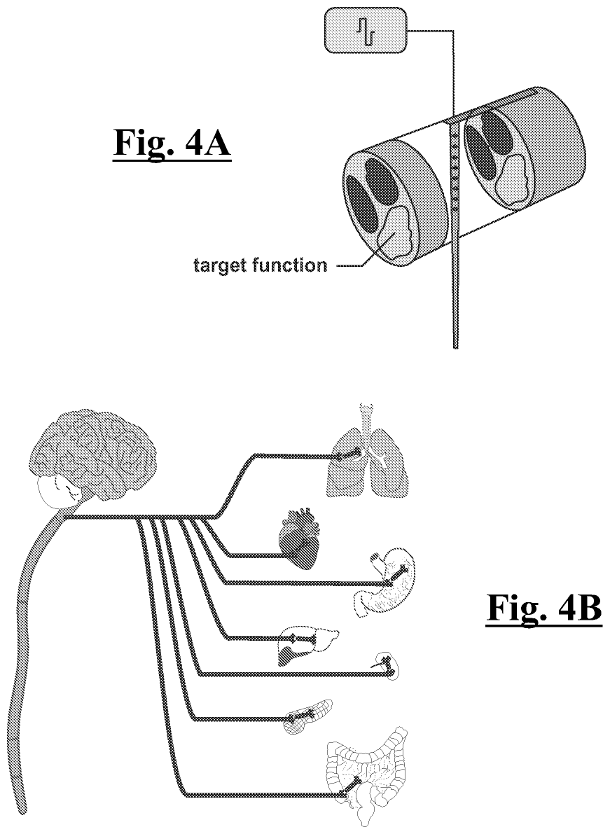

With reference to FIGS. 4A and 4B, it is then possible to use the information contained in the functional topography obtained to accurately stimulate the portion of the peripheral nerve correlated to the physiological signal, and therefore assigned to the modulation of the physiological parameter, on which one wishes to intervene. Consulting the functional topography graphed in

FIG. 3, for example, it is possible to determine a portion 410 which shows a strong correlation with the target physiological parameter on which it is desired to intervene. Therefore, in this case, the method according to the present invention provides for a selective stimulation on this area by the channels of the electrode 100, for example through an electrical stimulation by the channels most adjacent to this portion 410.

the foregoing description embodiments of the invention will so fully reveal the invention according to the conceptual point of view, so that others, by applying current knowledge, will be able to modify and/or adapt for various applications such embodiment without further research and without parting from the invention, and, accordingly, it is therefore to be understood that such adaptations and modifications will have to be considered as equivalent to the specific embodiments. The means and the materials to realise the different functions described herein could have a different nature without, for this reason, departing from the field of the invention. It is to be understood that the phraseology or terminology that is employed herein is for the purpose of description and not of limitation.

1. A method for determining the functional topography of a peripheral nerve of a user, said method requiring an electrode comprising a number n of channels cᵢ, with i=1, 2, . . . , n, wherein each channel cᵢ is in contact with said peripheral nerve at a respective contact point pᵢ, with i=1, 2, . . . , n,
said method comprising the steps of:
generating a model of a cross section S of said peripheral nerve where the area A of said cross section S comprises a number m of areas aⱼ, with j=1, 2, . . . , m;
computing a lead field matrix L=[R_{j,i}], wherein R_{j,i} is a value that describes the electrostatic relationship between an area aⱼ and a contact point pᵢ of said cross section S;
periodic acquisition, by said electrode, of a number n of voltage values V_{k,i} at instants tₖ, with k=1, 2, . . . , s, obtaining a voltage matrix V=[V_{k,i}], with i=1,2, . . . , n, where V_{k,i} is the voltage value determined by the channel cᵢ at the contact point pᵢ at the instant tₖ;
periodic acquisition, by at least one medical device, of a number r of values of physiological signals P_{k,h} of said user at instants tₖ, with k=1, 2, . . . , s, obtaining a matrix of the physiological signals P=P_{k,h}, with h=1, 2, . . . , r, where P_{k,h} is value of the h-th physiological signal determined at the instant tₖ;
computing a discrimination matrix D=[d_{h,i}], D being function of said matrices V=[V_{k,i}] and P=[P_{k,h}], where d_{h,i} is the discrimination coefficient which represents the correlation between the h-th physiological signal P_{k,h} and the i-th voltage value V_{k,i} referred to a same instant tₖ;
computing a spatial filtering matrix ϕ^{DBF}=[φ_{h,j}], φ_{h,j} being the localization index which represents the correlation between the h-th physiological signal and the area aⱼ of said cross section S;
for each h-th physiological signal, generating a functional topography of said peripheral nerve wherein each area aⱼ is graphically identified as a function of the corresponding value φ_{h,j} associated with it by said spatial filtering matrix ϕ^{DBF}.

2. The method for determining the functional topography of a peripheral nerve of a user, according to claim 1, wherein they are also provided the steps of:
filtering said voltage matrix V=[V_{k,i}] obtaining a filtered voltage matrix V̌=[V̌_{k,i}]=filt(V);
extracting features from said filtered voltage matrix V̌=[V̌_{k,i}] obtaining a neural data matrix X_{ENG},
and wherein said discrimination matrix D=[d_{h,i}] is function of said neural data matrix X_{ENG}.

3. The method for determining the functional topography of a peripheral nerve of a user, according to claim 2, where said step of filtering said voltage matrix V=[V_{k,i}] comprises the steps of:
for each channel cᵢ, defining a set Gᵢ comprising all the voltage values V_{k,i} taken at said channel cᵢ;
applying a filter on said set Gᵢ, obtaining a filtered set Ǧᵢ comprising filtered voltage values V̌_{k,i};
obtaining a filtered voltage matrix V̌=[V̌_{k,i}]=filt(V).

4. The method for determining the functional topography of a peripheral nerve of a user, according to claim 2, wherein said step of extracting features from said filtered voltage matrix V̌=[V̌_{k,i}] comprises the steps of:
defining a time window Δt_{\tilde{k}}=b*Δtₖ, with Δt_{\tilde{k}}=(t_{\tilde{k}+1}−t_{\tilde{k}}) and Δtₖ=(tₖ₊₁−tₖ), where b≥1 is a predetermined coefficient;
for each filtered set Ǧᵢ, selection of filtered voltage values V̌_{k,i} acquired in said time window Δt_{k̅}, obtaining a number s/b of subsets \tilde{G}_{k̅,i}, with \tilde{k}=1, 2, . . . , s/b, each subset \tilde{G}_{k,i} comprising a number b of filtered voltage values V̌_{k,i};
for each subset \tilde{G}_{k̅,i}, extraction of a number f of neural data arranged to define mathematical features of said subset \tilde{G}_{k,i}, obtaining a number n*f of neural data for each filtered set Ǧᵢ;
obtaining a neural data matrix X_{ENG}=[\tilde{V}_{k,i}], where \tilde{V}_{k̅,ī} is the ĩ-th neural datum extracted in the window Δt_{\overline{\tilde{k}}}, ĩ=1, 2, . . . , n*f.

5. The method for determining the functional topography of a peripheral nerve of a user, according to claim 1, wherein they are also provided the steps of:
filtering said matrix of the physiological signals P=[P_{k,h}] obtaining a filtered matrix of the physiological signals P̌=[P̌_{k,h}]=filt(P).
extracting features from said filtered matrix of the physiological signals P̌=[P̌_{k,h}] obtaining a functional data matrix X_{PHYSIO};
and wherein said discrimination matrix D=[d_{h,i}] is function of said functional data matrix X_{PHYSIO}.

6. The method for determining the functional topography of a peripheral nerve of a user, according to claim 5, wherein said step of filtering said matrix of the physiological signals P=[P_{k,h}] comprises the steps of:
for each h-th physiological signal, defining a set Gₕ comprising all the values of said h-th physiological signal P_{k,h} acquired;
applying a filter on said set Gₕ, obtaining a filtered set Ǧₕ comprising values of the filtered physiological signals P̌_{k,h};
obtaining a filtered matrix of the physiological signals P̌=[P̌_{k,h} k,h]=filt(P).

7. The method for determining the functional topography of a peripheral nerve of a user, according to claim 5, wherein said step of extracting features from said filtered matrix of the physiological signals P̌=P̌_{k,h} comprises the steps of:
defining a time window Δt_{\tilde{k}}=b*Δtₖ, with Δt_{\tilde{k}}=(t_{\tilde{k}+1}−t_{\tilde{k}}) and Δtₖ=(tₖ₊₁−tₖ), where b≥1 is a predetermined coefficient;
for each filtered set Ǧₕ, selection of values of filtered physiological signals P̌_{k,h} acquired in said time window Δtₖ, obtaining a number s/b of subsets \tilde{G}_{k,h}, with k=1,2, . . . , s/b, each subset \tilde{G}_{k,h} comprising a number b of filtered physiological signals P̌_{k,h};
for each subset \tilde{G}_{k,h}, extraction of a number w of functional data arranged to define mathematical features of said subset \tilde{G}_{k,h}, obtaining a number n*w of functional data for each filtered set Ǧₕ;
obtaining a functional data matrix X_{PHYSIO}=[\tilde{P}_{k,h}], where \tilde{P}_{k,h} is the \tilde{h}-th functional datum extracted in the window Δtₖ, \tilde{h}=1, 2, . . . , n*w.

8. The method for determining the functional topography of a peripheral nerve of a user, according to claim 2, wherein said step of computing said discrimination matrix D is obtained solving the system:
$$\{ \begin{matrix}{X}_{ENG} = {X}_{PHYSIO} D + ε \\ D = {( {X}_{PHYSIO}^{T} {C}_{ε}^{- 1} {X}_{PHYSIO} )}^{- 1} {X}_{PHYSIO}^{T} {C}_{ε}^{- 1} {X}_{ENG} \\ {C}_{ε} = E \{ ( ε - {η}_{ε} ) {( ε - {η}_{ε} )}^{T} \}\end{matrix}$$
where C_{ε} is the error covariance matrix,
E is the expected value operator,
ε is the error matrix in which the residuals of the predictive model are present.

9. The method for determining the functional topography of a peripheral nerve of a user, according to claim 2, wherein said step of computing said discrimination matrix D is obtained by the equation:
$${d}_{h , i} = corr ( {X}_{{PHYSIO}_{k , h}} , {X}_{{ENG}_{k , i}} ) = \frac{{σ}_{{X}_{{PHYSIO}_{k , h}} , {X}_{{ENG}_{k , i}}}}{{σ}_{{X}_{{PHYSIO}_{k , h}}} {σ}_{{X}_{{ENG}_{k , i}}}}$$
where σ_{X}_{_{PHYSIOk,h}}_{,X}_{_{ENGk,i}} is the covariance of the variables X_{PHYSIO}_{_{k,h}} and X_{ENG}_{_{k,i}}_{,}
σ_{X}_{_{PHYSIOk,h}}/σ_{X}_{_{ENGk,i}} is the standard deviation of X_{PHYSIO}_{_{k,h}}/X_{ENG}_{_{k,i}}.

10. The method for determining the functional topography of a peripheral nerve of a user, according to claim 1, wherein said step of computing said spatial filtering matrix ϕ^{DBF} is obtained according to the equation:
ϕ^{DBF}=DL⁺
with L⁺=(L^{T}L)⁻¹L^{T}.

11. The method for determining the functional topography of a peripheral nerve of a user, according to claim 1, wherein said step of computing said spatial filtering matrix ϕ^{DBF} is obtained according to the equation:
ϕ^{DBF}=DL_{Λ}⁻
with L_{Λ}⁺=(L^{T}L)⁻¹L^{T}Λ,
where Λ=[Λ_{j,j}] is the spatial information matrix, being Λ_{j,j}=1 when it is known that the area aⱼ corresponds to a nonzero value of φ_{h,j}.

12. The method for determining the functional topography of a peripheral nerve of a user, according to claim 1, wherein said step of computing said spatial filtering matrix ϕ^{DBF} is obtained according to the equation:
$${ϕ}^{DBF} = D {\hat{L}}_{Λ}^{+}$$
$$with {L}_{Λ}^{+} = {( {L}^{T} Λ L )}^{- 1} {L}^{T} Λ and {\hat{L}}_{Λ}^{+} [ : , j ] ← \frac{{\hat{L}}_{Λ}^{+} [ : , j ]}{{‖ Λ {LL}_{Λ}^{+} [ : , j ] ‖}_{2}} ,$$
where Λ=[Λ_{j,j}] is the spatial information matrix, being Λ_{j,j}=1 when it is known that the area aⱼ corresponds to a nonzero value of φ_{h,j}.

13. The method for determining the functional topography of a peripheral nerve of a user, according to claim 1, wherein said step of generating a functional topography of said peripheral nerve is obtained by associating a plurality of numerical ranges of said values φ_{h,j} to respective colours or colour shades.

14. The method for determining the functional topography of a peripheral nerve of a user, according to claim 1, wherein a step is also provided of electrically stimulating, by means of said electrode, at least one area aⱼ of said cross section S, in order to vary the physiological signal P_{k,h} of said user associated with said area aⱼ.
